(19) United States
(12) Patent Application Publication
(10) Pub. No.: US 2007/0088260 A1
(43) Pub. Date: Apr. 19, 2007

(54) Syringe with safety cap

(76) Inventor: Kai-Hsiang Peng, Daliao Township (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/242,840

(22) Filed: Oct. 5, 2005

(51) Int. Cl. A61M 5/00

(52) U.S. Cl. 604/110

(57) ABSTRACT

A syringe includes a barrel with a plunger movably inserted therein and a needle set is connected to a neck extending from an end of the barrel and includes a needle. A cone-shaped member extends radially outward from the needle. A cap has an insertion end for receiving the needle and a close end. A positioning device is connected to the cap and includes a plurality of first plates and second plates extending inward from the inner periphery of the cap. After use, the needle penetrates through holes enclosed by the first and second plates and the cone-shaped member is positioning between the first and second plates. Due to the holding of the cone-shaped member between the first and the second plates, the cap can not be removed from the needle and enhance the safety to recycle the syringes.

### FIELD OF THE INVENTION

The present invention relates to a syringe with a safety cap which is able to firmly connect to the needle after use.

### BACKGROUND OF THE INVENTION

A conventional syringe 1 is shown in FIG. 1 and generally includes a barrel 11 with a passage 111 defined therethrough and a plunger 13 with a stopper is movably inserted into the passage 111 so as to push medicine out from a needle set 12 which includes a locking tip 121 and a needle 122. The locking tip 121 is fixedly connected to a neck on a front end of the barrel 11. The neck has a through hole 112 which is in communication with the passage of the barrel 11. A cap 14 is movably mounted to the locking tip 121 so as to protect users from being injury by the exposed needle 122. When in use, the user remove the cap 14 to proceed injection, and the cap 14 is re-cap to the locking tip 121 after use. However, the user often be hurt by the needle 122 when re-capping the cap 14 and the cap 14 is easily to be removed from the locking tip 121 after the syringe is discarded. The janitors might be hurt by the needles 122 without the caps 14.

The present invention intends to provide a safety cap for syringe, wherein the needle includes a cone-shaped member and the cap includes a plurality of plates which hold the cone-shaped member on the needle after the syringe is used so that the cap can not be removed from the needle.

### SUMMARY OF THE INVENTION

The present invention relates to a syringe that comprises a barrel with a plunger movably inserted into the passage defined in the barrel, a needle set having a locking tip which is connected to a neck extending from an end of the barrel and a needle is connected to the locking tip. A cone-shaped member extends radially outward from the needle. A cap has an insertion end and a close end. A positioning device is connected to the cap and includes a plurality of first plates and second plates extending inward from the inner periphery of the cap. The first plates and the second plates are arranged to enclose two respective holes so that the needle can slidably penetrate therethrough. A gap is defined between the first and second plates so that the cone-shaped member on the needle is held in the gap between the first and second plates.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a cross sectional view to show a conventional syringe;

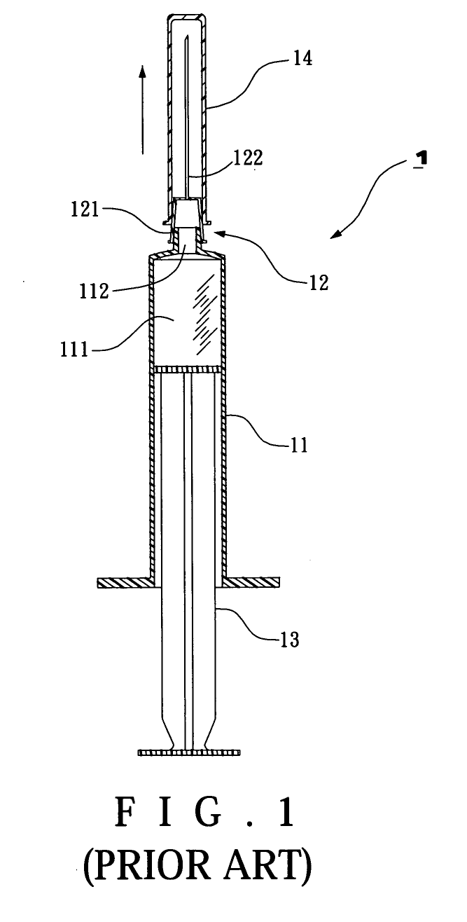

FIG. 2 is an exploded view to show the syringe of the present invention;

FIGS. 3 and 4 show that the needle is inserted into the cap and the cone-shaped member is held between the first and second plates in the cap;

FIG. 5 is an exploded view to show a second embodiment of the syringe of the present invention;

FIG. 6 shows that the cap is mounted to the locking tip and the needle is received in the cap before the syringe is used;

FIG. 7 shows that that the needle is inserted into the cap and the cone-shaped member is held between the first and second plates in the cap;

FIG. 8 shows a weight is received in the flange of the cap, and

FIG. 9 shows a magnet is received in the flange of the cap.

### DETAILED DESCRIPTION OF THE PREFERRED EMBODIMENT

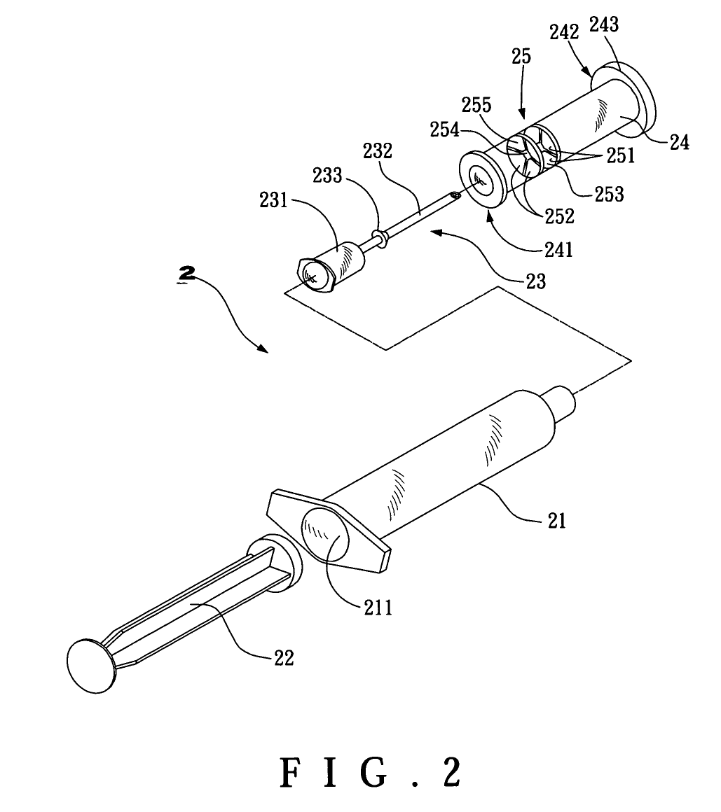

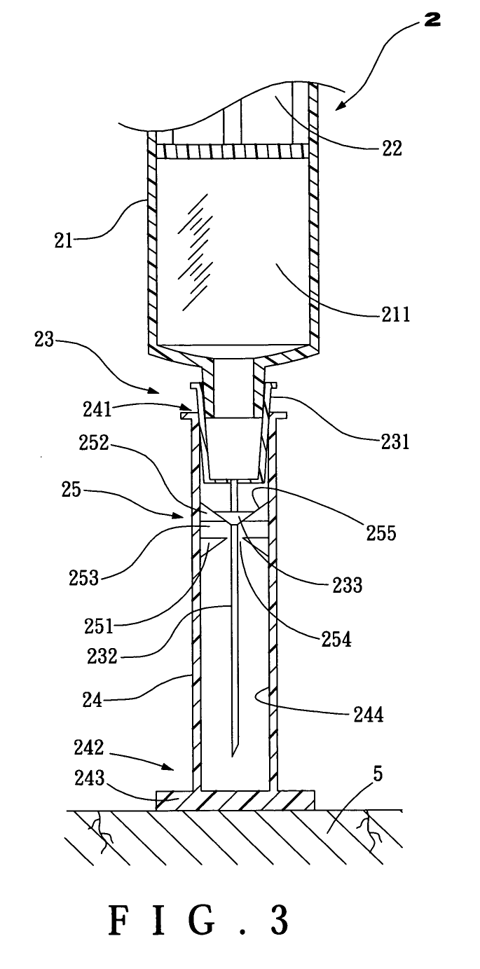

Referring to FIGS. 2 and 3, the syringe 2 of the present invention comprises a barrel 21 with a passage 211 defined therethrough and a plunger 22 has one end movably inserted into the passage 211 from a first open end of the barrel 21. A neck extends from a second end of the barrel and a needle set 23 is connected to the neck. The needle set 23 has a locking tip 231 which is connected to the neck and a needle 232 is connected to the locking tip 231. A cone-shaped member 233 extends radially outward from the needle 232.

A cap 24 has an insertion end 241 and a close end 242, wherein the close end 242 includes a flange 243 extending radially outward therefrom and a diameter of the flange 243 is larger than a diameter of the insertion end 241. A positioning device 25 is connected to an inner periphery 244 of the cap 24 and includes a plurality of first plates 251 and second plates 252 extending inward from the inner periphery 244 of the cap 24. The first plates 251 and the second plates 252 are arranged to enclose two respective holes 254, and a gap 253 is defined between the first and second plates 251, 252. The second plates 252 are located close to the insertion end 241 and each of the second plates 252 has an inclined surface 255 facing the insertion end 241.

After the syringe is used, the cap 24 is re-capped to the locking tip 231 and the needle 232 is received in the cap 24. The user puts the flange 243 on the table 5 and pushes the barrel 21 downward so that the cone-shaped member 233 slides over the inclined surfaces 255 and held between the first and second plates 251, 252 in the gap 253. The cap 24 is then securely mounted onto the locking tip 231 and the needle 232 is enclosed by the cap 24.

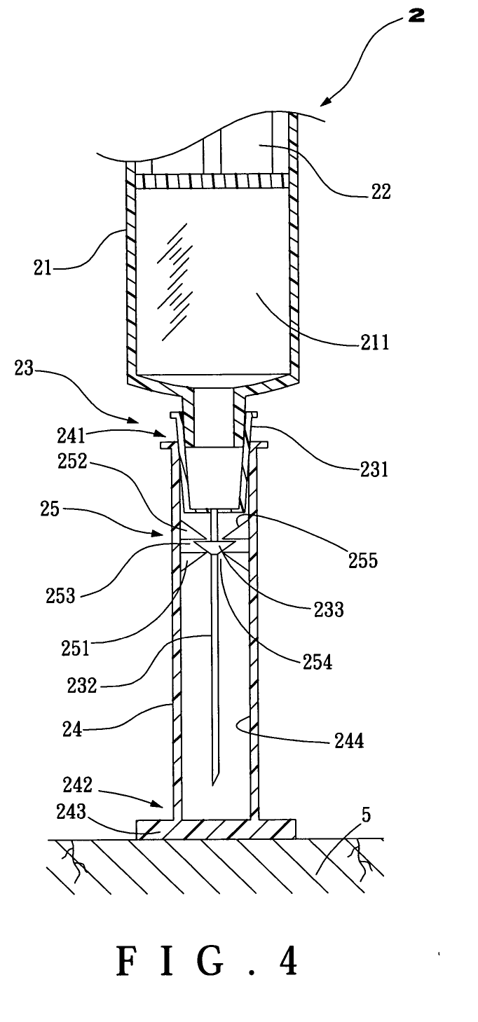

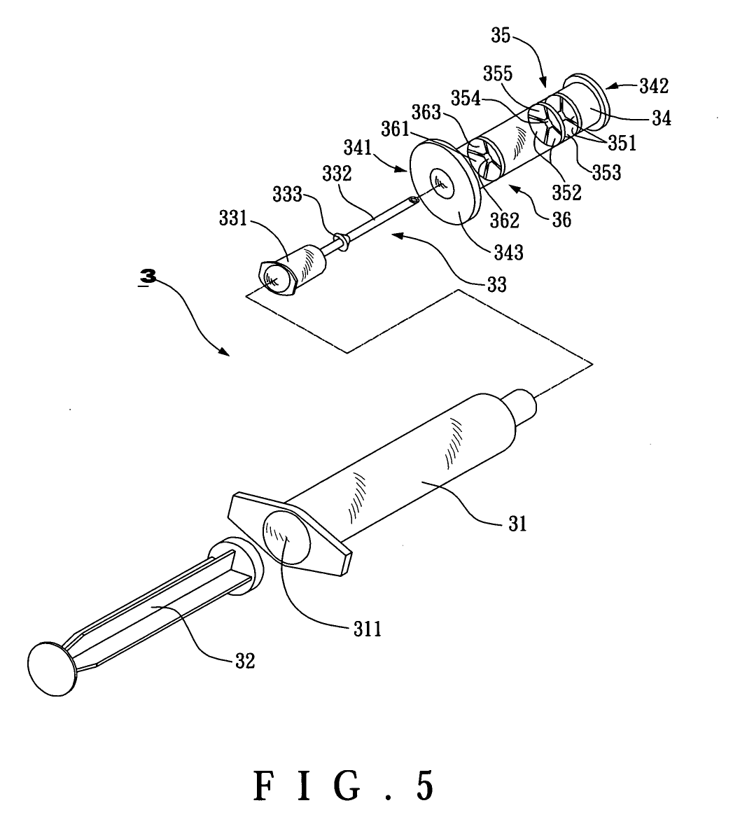

FIGS. 5 and 6 show a second embodiment of the present invention wherein the syringe 3 includes a barrel 31 with a passage 311 defined therethrough and a plunger 32 has one end movably inserted into the passage 211. A needle set 33 has a locking tip 331 which is connected to a neck extending from an end of the barrel 31 and a needle 332 is connected to the locking tip 331. A cone-shaped member 333 extends radially outward from the needle 332.

A cap 34 has a first end 341 and a second end 342, The first end 341 includes a flange 343 extends radially outward therefrom and a diameter of the flange 343 is larger than a diameter of the second end 342. A first positioning device 35 and a second positioning device 36 are respectively connected to an inner periphery 344 of the cap 34. The first positioning device 35 includes a plurality of first plates 351 and second plates 352 extending inward from the inner periphery 344 of the cap 34. The first plates 351 and the second plates 352 are arranged to enclose two respective holes 354. A gap 353 is defined between the first and second plates 351, 352. The second positioning device 36 is located close to the first end 341 of the cap 34 and includes third plates 361 extending inward from the inner periphery 344 of the cap 34. The third plates 361 are arranged to enclose a hole 362. The second plates 352 are located close to the first end 341 of the cap 34 than the first plates 351 and each of the second plates 352 has an inclined surface 355. The third plates 361 are located close to the first end 341 of the cap 34 and each of the second plates 352 has an inclined surface 363 facing the first end 341 so that the cone-shaped member 333 is in contact with the inclined surfaces 363 of the third plates 361 when the syringe 3 has not yet used.

Besides, to prevent mixing the used syringe and unused syringe, the surface of cup in the position of first plates and second plates are marked on different colors which indicate the using status of the syringe.

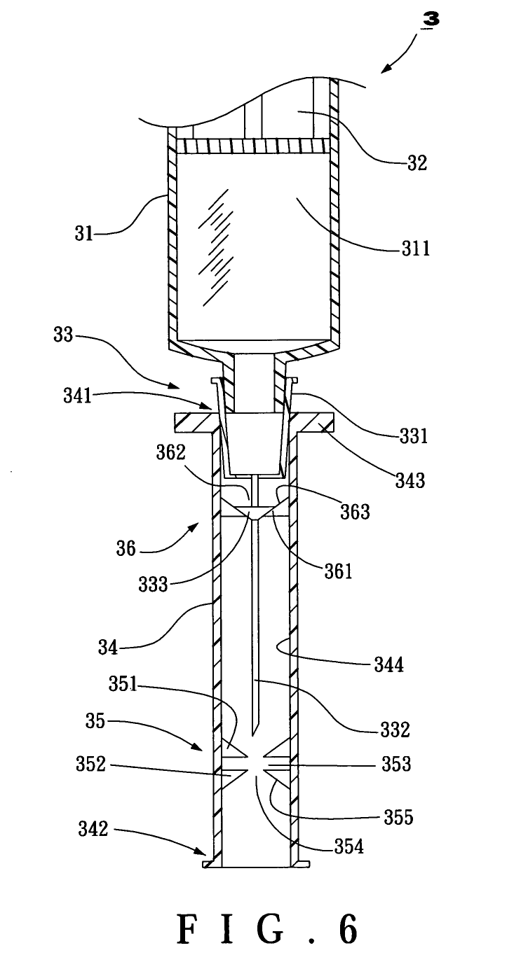

As shown in FIG. 7, after the syringe 3 is used, the cap 34 is re-capped to the locking tip 331 and the needle 332 is received in the cap 34. The user puts the flange 343 on the table 6 and pushes the barrel 31 downward so that the cone-shaped member 333 slidably penetrates through the hole 354 enclosed by the second plates 352 and the cone-shaped member 333 slides over the inclined surfaces 355 and held between the first and second plates 351, 352 in the gap 353. The cap 24 is then securely mounted onto the locking tip 331 and the needle 332 is enclosed by the cap 34.

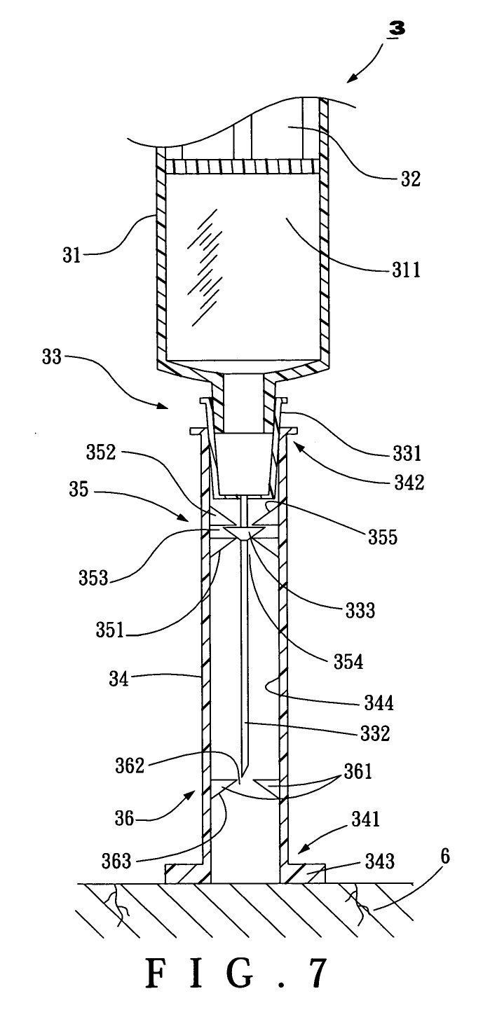

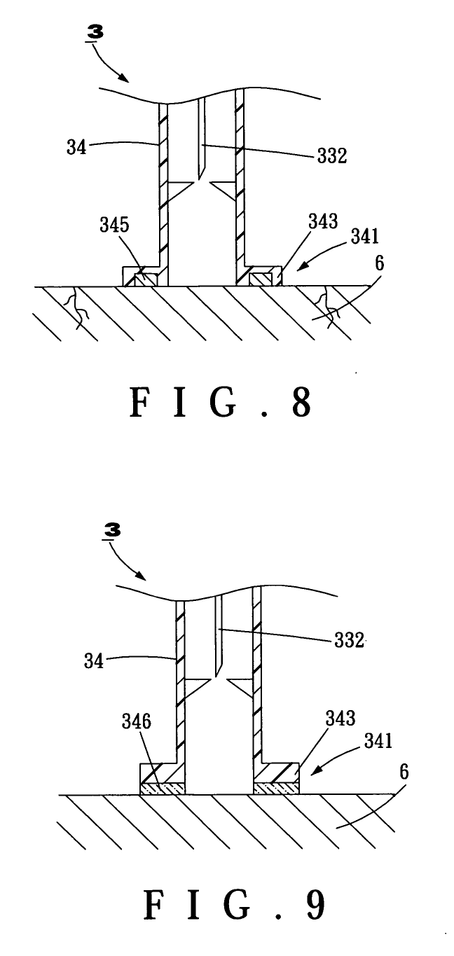

It is noted that a weight 345 can be received in the flange 343 as shown in FIG. 8 to assist the user to put the flange 343 on the table 6. A magnet 346 can also be received in the flange 343 as shown in FIG. 9 so that if the table 6 has a metal top, the magnet 346 assists the user to firmly stand the cap 34 on the table 6.

As indicated above, the invention has the following advantages:
1. Because the design of cone-shaped member and plates, the cup is hardly removed from the needle after using and inserting the needle into the cap even trying to separate them with force. In case the syringe is missing, the needle is difficult to pull out from the cup so that accidence can be prevented.
2. Because of the different colors of marks on the surface of cup in the position of first plates and second plates, the medical personnel easily understand the using status of syringe and prevent using the used syringe again.
3. Because of the flange, the cap firmly stands on the table which is convenient for medical personnel to mount the cap with single hand and prevent to get hurt by the needle.

While we have shown and described the embodiment in accordance with the present invention, it should be clear to those skilled in the art that further embodiments may be made without departing from the scope of the present invention.

What is claimed is:

1. A syringe comprising:
a barrel with a passage defined therethrough and a plunger having one end movably inserted into the passage;
a needle set having a locking tip which is connected to a neck extending from an end of the barrel and a needle connected to the locking tip, a cone-shaped member extending radially outward from the needle; and
a cap having an insertion end and a close end, a positioning device connected to an inner periphery of the cap and including a plurality of first plates and second plates extending inward from the inner periphery of the cap, the first plates and the second plates being arranged to enclose two respective holes, a gap defined between the first and second plates.

2. The syringe as claimed in claim 1, wherein the second plates are located close to the insertion end and each of the second plates has an inclined surface facing the insertion end so that the cone-shaped member slidably penetrates through the hole enclosed by the second plates.

3. The syringe as claimed in claim 1, wherein the close end includes a flange extending radially outward therefrom and a diameter of the flange is larger than a diameter of the insertion end.

4. The syringe as claimed in claim 3 further comprising a weight received in the flange.

5. The syringe as claimed in claim 3 further comprising a magnet received in the flange.

6. A syringe comprising:
a barrel with a passage defined therethrough and a plunger having one end movably inserted into the passage;
a needle set having a locking tip which is connected to a neck extending from an end of the barrel and a needle connected to the locking tip, a cone-shaped member extending radially outward from the needle, and
a cap having a first end and a second end, a first positioning device and a second positioning device connected to an inner periphery of the cap, the first positioning device including a plurality of first plates and second plates extending inward from the inner periphery of the cap, the first plates and the second plates being arranged to enclose two respective holes, a gap defined between the first and second plates, the second positioning device located close to the first end of the cap and including third plates extending inward from the inner periphery of the cap, the third plates being arranged to enclose a hole.

7. The syringe as claimed in claim 6, wherein the second plates are located close to the first end of the cap than the first plates and each of the second plates has an inclined surface so that the cone-shaped member slidably penetrates through the hole enclosed by the second plates.

8. The syringe as claimed in claim 6, wherein the third plates are located close to the first end of the cap and each of the second plates has an inclined surface facing the first end so that the cone-shaped member is in contact with the inclined surfaces of the third plates.

9. The syringe as claimed in claim 6, wherein the first end includes a flange extending radially outward therefrom and a diameter of the flange is larger than a diameter of the second end.

10. The syringe as claimed in claim 9 further comprising a weight received in the flange.

11. The syringe as claimed in claim 9 further comprising a magnet received in the flange.
